(19) United States
(12) Patent Application Publication
(10) Pub. No.: US 2019/0076835 A1
(43) Pub. Date: Mar. 14, 2019

(54) PROCESSSES USING MULTIFUNCTIONAL CATALYSTS

(71) Applicant: Novomer, Inc., Waltham, MA (US)

(72) Inventor: Sadesh H. Sookraj, Cambridge, MA (US)

(73) Assignee: Novomer, Inc.

(21) Appl. No.: 15/701,410

(22) Filed: Sep. 11, 2017

(51) Int. Cl. B01J 35/00; B01J 23/28; B01J 23/26; B01J 23/18; B01J 21/08; B01J 21/04; B01J 31/18; B01J 31/22; C07C 67/37; C08G 63/82

(52) U.S. Cl. CPC B01J 35/0006; B01J 23/28; B01J 23/26; B01J 23/18; B01J 21/08; B01J 2231/321; B01J 31/1815; B01J 31/2213; C07C 67/37; C08G 63/823; B01J 2231/14; B01J 21/04

(57) ABSTRACT

The present invention is directed to processes for catalyzing two or more chemical reactions with a multifunctional catalyst in a reaction vessel. The processes include steps for introducing one or more reagents to a reaction vessel containing a multifunctional catalyst; contacting the one or more reagents with a first portion of the multifunctional catalyst to produce an intermediate; contacting the intermediate with a second portion of the multifunctional catalyst to produce a product; and removing the product from the reaction vessel. In certain embodiments, the multifunctional catalyst may have a first portion with carbonylation functionality for catalyzing the production of a beta-lactone intermediate from an epoxide reagent and a carbon monoxide reagent. In certain embodiments, the multifunctional catalyst may have a second portion with a functionality suitable for polymerization, co-polymerization, and/or modification of a beta-lactone intermediate. In preferred embodiments, the first portion and second portion are bonded to a heterogenous support.

### FIELD OF THE INVENTION

The present invention generally is directed to processes for catalyzing two or more chemical reactions with a multifunctional catalyst in a reaction vessel. More specifically, the processes comprise catalyzing carbonylation of an epoxide in the presence of carbon monoxide and to form a beta-lactone intermediate and subsequently reacting the beta-lactone intermediate. Advantageously, embodiments of the present invention may catalyze carbonylation, polymerization, copolymerization and/or modification in one reaction vessel.

### BACKGROUND OF THE INVENTION

A catalyst is an atom or molecule which may alter a chemical reaction. Generally, the catalyst may provide an alternative mechanism for a chemical reaction with a different transition state and/or activation energy. A portion of a catalyst, such as an atom, ion, or molecule, may have a particular functionality. For example, one portion of a catalyst may have a Lewis acid functionality but another portion of the catalyst may have a Lewis base functionality. A catalyst having multiple functionalities is termed a multifunctional catalyst for the purposes of this invention.

A catalyst may be characterized as a heterogenous catalyst or a homogenous catalyst depending on whether the catalyst is present in the same phase state as other chemical reagents. A homogenous catalyst will generally be in the same phase state and miscible with other chemical reagents. A heterogenous catalyst will generally be in a different phase state and immiscible with the other chemical reagents.

Certain catalysts, termed carbonylation catalysts for the purposes of this invention, may catalyze the carbonylation of epoxides, aziridines, thiiranes, oxetanes, lactones, lactams, and analogous compounds to produce ring-expanded products. Some conventional carbonylation catalysts comprise a metal carbonyl-Lewis acid catalyst such as those described in U.S. Pat. Nos. 6,852,865, 5,310,948; 7,420,064; and 5,359,081. Many conventional carbonylation catalysts are homogenous catalysts which must be separated from the ring-expanded products for further reaction resulting in extra process steps, manufacturing costs, and overly complex production systems.

There exists need for processes providing for carbonylation and subsequent conversion of a beta-lactone in the same reaction vessel. The present invention satisfies this need by providing for carbonylation and subsequent conversion of a beta-lactone in the same reaction vessel with a multifunctional catalyst.

### SUMMARY OF THE INVENTION

There he present invention is directed to processes comprising catalyzing two or more chemical reactions with a multifunctional catalyst in the same reaction vessel. Chemical reagents may be introduced to the reaction vessel to contact and react with a first portion of the multifunctional catalyst having a particular functionality to produce an intermediate molecule. The intermediate molecule may react with a second portion and/or another portion of the multifunctional catalyst having a particular functionality to produce a product and/or another intermediate molecule. Advantageously, the processes of the present invention reduce costs associated with facilities, equipment, safety, and personnel by catalyzing two or more chemical reactions with a multifunctional catalyst in the same reaction vessel.

In preferred embodiments, the processes of the present invention provide for carbonylation. The multifunctional catalyst may have a first portion with a carbonylation functionality. The first portion with the carbonylation functionality may comprise a metal carbonyl-Lewis acid catalyst. In preferred embodiments, an epoxide reagent and a carbon monoxide reagent may be introduced to a reaction vessel containing a multifunctional catalyst with a first portion having a carbonylation functionality. The epoxide reagent and carbon monoxide reagent may react with the first portion having a carbonylation functionality to produce a beta-lactone intermediate. In some embodiments, the beta-lactone intermediate may react with the first portion having a carbonylation functionality to produce a succinic anhydride intermediate and/or a succinic anhydride product wherein the succinic anhydride intermediate may react with a second portion of the multifunctional catalyst and the succinic anhydride product may be removed from the reaction vessel.

In preferred embodiments, the processes of the present invention provide for polymerization. The multifunctional catalyst may have a first portion with a carbonylation functionality and a second portion having ring-opening polymerization functionality. The second portion having ring-opening polymerization functionality may comprise a carboxylate salt of an organic cation, metal-containing complexes, protonated amine, quaternary ammonium salt, guanidinium group, and/or optionally substituted nitrogen-containing heterocycle to name a few. The epoxide reagent and carbon monoxide reagent may be introduced to a reaction vessel and react with the first portion having a carbonylation functionality to produce a beta-lactone intermediate. The beta-lactone may react with the second portion having ring-opening polymerization functionality to produce a polylactone intermediate and/or polylactone product wherein the polylactone intermediate may react with a third portion of the multifunctional catalyst and the polylactone product may be removed from the reaction vessel.

In preferred embodiments, the processes of the present invention provide for co-polymerization and/or modification. The multifunctional catalyst may have a first portion with a carbonylation functionality and one or more other portions with co-polymerization and/or modification functionality. The one or more other portions may comprise condensation polymerization catalysts, oxide catalysts, precious metal catalysts, and/or combinations therein. In preferred embodiments providing for co-polymerization and/or modification, one or more reagents may be introduced to the reaction vessel for reaction with a beta-lactone intermediate. In certain embodiments, the beta-lactone intermediate may be co-polymerized with.

Advantageously, the processes of the present invention provide for the formation of beta-lactones and subsequent conversion of the beta-lactones without the need for multiple catalysts. Some advantages of the present invention are decreased equipment and handling requirements associated with changing catalysts, transferring intermediates to subsequent reaction vessels, and handling beta-lactones.

While this disclosure is susceptible to various modifications and alternative forms, specific exemplary embodiments thereof have been shown by way of example in the drawings and have herein been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

### DETAILED DESCRIPTION OF EMBODIMENTS

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The present invention is directed to processes comprising steps for catalyzing two or more chemical reactions with a multifunctional catalyst in a reaction vessel. In certain embodiments, the processes may include a reaction vessel configured so that the processes of the present invention may be performed continuously. In some embodiments, the reaction vessel may be configured as a fixed-bed continuous reactor or fluidized-bed continuous reactor. The reaction vessel comprises mainly metal materials, such as carbon steel and stainless steel, and use of stainless steel is preferable. The processes of the present invention may include reaction vessels having one or more reaction zones, inlets, outlets, heat exchange surfaces, filters, and/or mixers.

In preferred embodiments, the processes comprise the steps: introducing one or more reagents to a reaction vessel; contacting the one or more reagents with a first portion of a multifunctional catalyst to produce an intermediate; contacting the intermediate with a second portion of a multifunctional catalyst to produce a product; and removing the product from the reaction vessel.

In certain preferred embodiments, a feed stream comprising one or more reagents may be introduced to a reaction zone of the reaction vessel containing a multifunctional catalyst. The feed stream may be introduced as a gas, liquid, and/or solid. The feed stream may be introduced at lower than ambient temperature, ambient temperature, or higher than ambient temperature. The feed stream may be introduced at lower than ambient pressure, ambient pressure, or higher than ambient pressure. In some embodiments, it may be preferable to introduce a first reagent to the reaction vessel before introducing a second reagent to the reaction vessel. In certain embodiments, the feed stream may comprise two or more feed streams introduced to the reaction vessel at different phases, temperatures, pressures, and/or times.

In certain preferred embodiments, one or more reagents may diffuse to contact a first portion of the multifunctional catalyst and produce an intermediate. In certain embodiments, one or more reagents may diffuse as a gas to contact the first portion of a solid phase multifunctional catalyst. For example, one or more reagents introduced to a reaction vessel as liquid at ambient temperature may be heated to gas phase and diffuse to contact the first portion of the solid phase multifunctional catalyst. In another example, one or more reagents introduced to a reaction vessel as a gas may diffuse to contact the first portion of the solid phase multifunctional catalyst. In certain embodiments, one or more reagents may diffuse as a liquid to contact the first portion of a solid phase multifunctional catalyst. For example, one or more liquid phase reagents may be introduced to a reaction vessel above a solid phase multifunctional catalyst and diffuse to contact the first portion under the force of gravity.

In certain preferred embodiments, an intermediate may diffuse to contact a second portion of the multifunctional catalyst and produce a product. In certain embodiments, the intermediate may diffuse as a gas to contact the second portion of a solid phase multifunctional catalyst. For example, an intermediate in liquid phase may be heated to gas phase and diffuse to contact the second portion of the solid phase multifunctional catalyst. In certain embodiments, an intermediate may diffuse as a liquid to contact the second portion of a solid phase multifunctional catalyst. For example, an intermediate may diffuse to contact the second portion under the force of gravity. In some embodiments, the intermediate may be cooled from gas phase to liquid phase and diffuse to contact the second portion under the force of gravity.

Preferably, the product may be removed from the reaction vessel in a liquid phase or a gas phase. In preferred embodiments, the multifunctional catalyst may remain in the reaction vessel in a solid phase. In certain preferred embodiments, a product in liquid phase may be heated to gas phase and removed from the reaction vessel. In certain other preferred embodiments, a product in gas phase may be cooled to liquid phase and removed from the reactor. In certain embodiments, the product may be separated from the multifunctional catalyst, impurities, unreacted reagents, or any other undesired materials by phase separation, distillation, ion exchange filtration, and/or molecular sieve filtration.

The one or more reagents include an epoxide reagent and a carbon monoxide reagent. The epoxide reagent and carbon monoxide reagent may have high biobased carbon content wherein Bio-based content=[Bio (Organic) Carbon]/[Total (Organic) Carbon]100%, as determined by ASTM D6866 (Standard Test Methods for Determining the Bio-based Content of Solid, Liquid, and Gaseous Samples Using Radiocarbon Analysis). The epoxide reagent and carbon monoxide reagent with high biobased carbon content may be produced, from biologically sourced, renewable, recycled, and/or sustainable sources of carbon such as bio-mass derived carbon, carbon waste streams, and carbon from municipal solid waste. The epoxide reagent and carbon monoxide reagent with high biobased carbon content may have a biobased content of at least 10% and preferably at least 20%, more preferably at least 50%. A biobased content of at least 90%, at least 95%, at least 99%, or 100% is particularly preferred. The epoxide reagents and carbon monoxide reagents with high biobased content may have certain organic impurities. In some embodiments, multifunctional catalysts may include a first portion selective for epoxides and carbon monoxide over other organic impurities.

The processes of the present invention include multifunctional catalysts configured to provide for carbonylation of an epoxide reagent with a carbon monoxide reagent. The multifunctional catalyst has a first portion with a carbonylation functionality. In certain preferred embodiments, the first portion with the carbonylation functionality may comprise a metal carbonyl-Lewis acid. In preferred embodiments, an epoxide reagent and a carbon monoxide reagent may be introduced to a reaction vessel containing a multifunctional catalyst with a first portion having a carbonylation functionality. The epoxide reagent and carbon monoxide reagent may contact and react with the first portion having a carbonylation functionality to produce a beta-lactone intermediate. In some embodiments, the beta-lactone intermediate may react with the first portion having a carbonylation functionality to produce a succinic anhydride intermediate and/or a succinic anhydride product wherein the succinic anhydride intermediate may react with a second portion of the multifunctional catalyst and the succinic anhydride product may be removed from the reaction vessel. In other embodiments, the beta-lactone intermediate may react with a second portion of the multifunctional catalyst having a carbonylation functionality selective to reacting with the beta-lactone intermediate to produce a succinic anhydride intermediate and/or a succinic anhydride product.

The carbonylation functionality of a multifunctional catalyst may utilize a metal carbonyl-Lewis acid moiety such as those described in U.S. Pat. No. 6,852,865. In certain embodiments, the carbonylation functionality includes one or more molecules of the carbonylation catalysts disclosed in U.S. patent application Ser. Nos. 10/820,958; and 10/586,826. In certain embodiments, the carbonylation functionality includes one or more molecules of the catalysts disclosed in U.S. Pat. Nos. 5,310,948; 7,420,064; and 5,359,081. The entirety of each of the preceding references is incorporated herein by reference. In certain preferred embodiments, the metal carbonyl-Lewis acid moiety is bonded or otherwise tethered to a heterogenous support comprising insoluble siliceous material or an insoluble carbon matrix.

Table 1 illustrated below includes Column A directed to a non-exhaustive list of epoxides which may undergo carbonylation to produce beta-lactone intermediates according to the processes of the present invention and Column B directed to a non-exhaustive list of the beta-lactone intermediates.

TABLE 1

| Column A | Column B |
|---|---|
| 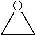 | 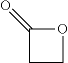 |
| 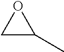 | 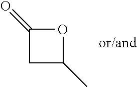 |
| | 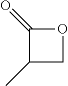 |
| 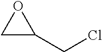 | 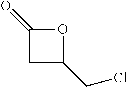 |
| 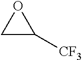 | 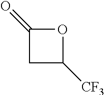 |
| 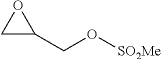 | 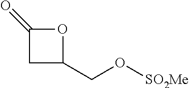 [SMILES: CS(=O)(=O)OCC1CC(=O)O1] |
| 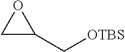 [SMILES: CC(C)(C)[Si](C)(C)OCC1CO1] | 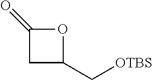 [SMILES: CC(C)(C)[Si](C)(C)OCC1CC(=O)O1] |
| 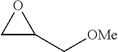 | 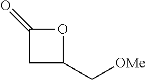 |
| 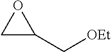 | 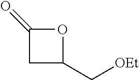 |
| 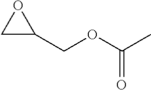 | 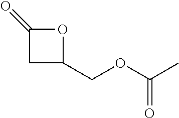 |
| 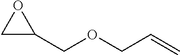 | 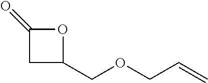 |
| 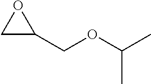 | 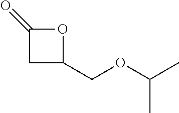 |
| 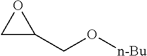 | 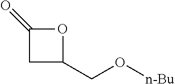 |
| 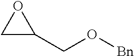 [SMILES: c1ccc(COCC2CO2)cc1] | 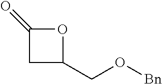 [SMILES: O=C1CC(COCc2ccccc2)O1] |
| 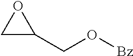 | 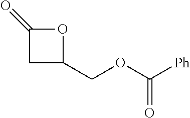 [SMILES: O=C1CC(COC(=O)c2ccccc2)O1] |
| 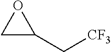 | 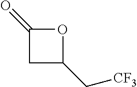 |
| 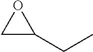 | 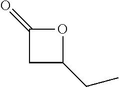 |
| 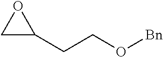 [SMILES: c1ccc(COCCC2CO2)cc1] | 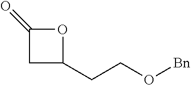 [SMILES: O=C1CC(CCOCc2ccccc2)O1] |
| 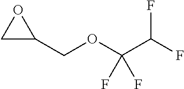 [SMILES: FC(F)C(F)(F)OCC1CO1] | 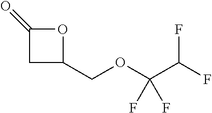 [SMILES: O=C1CC(COC(F)(F)C(F)F)O1] |
| 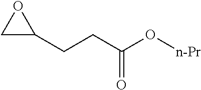 | 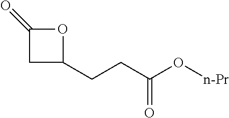 |
| 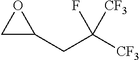 [SMILES: FC(F)(F)C(F)(CC1CO1)C(F)(F)F] | 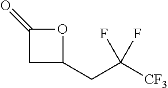 [SMILES: O=C1CC(CC(F)(F)C(F)(F)F)O1] |
| 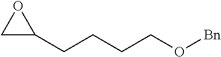 [SMILES: c1ccc(COCCCCC2CO2)cc1] | 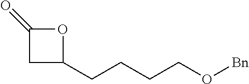 [SMILES: O=C1CC(CCCCOCc2ccccc2)O1] |
| 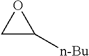 | 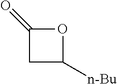 |
| 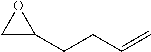 | 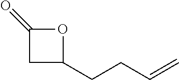 |
| 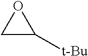 | 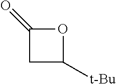 |
| 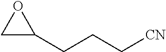 | 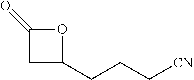 |
| 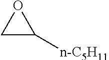 | 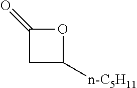 |
| 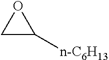 | 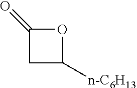 |
| 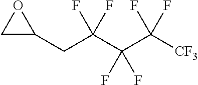 [SMILES: FC(F)(F)C(F)(F)C(F)(F)C(F)(F)CC1CO1] | 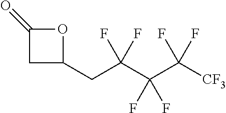 [SMILES: O=C1CC(CC(F)(F)C(F)(F)C(F)(F)C(F)(F)F)O1] |
| 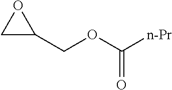 | 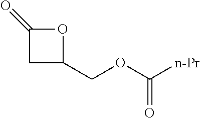 |
| 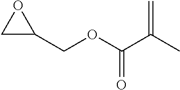 | 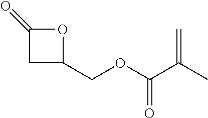 |
| 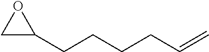 | 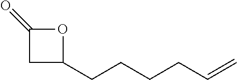 |
| 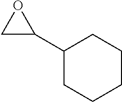 | 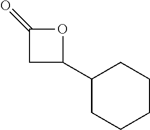 [SMILES: O=C1CC(C2CCCCC2)O1] |
| 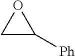 | 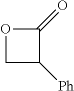 [SMILES: O=C1OCC1c1ccccc1] |
| 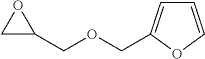 [SMILES: c1coc(COCC2CO2)c1] | 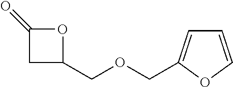 [SMILES: O=C1CC(COCc2ccco2)O1] |
| 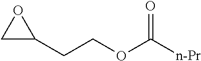 | 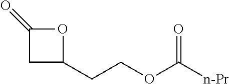 |
| 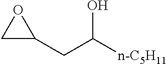 | 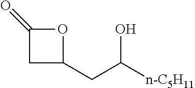 |
| 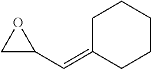 | 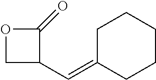 [SMILES: O=C1OCC1C=C1CCCCC1] |
| 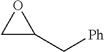 | 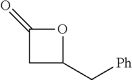 [SMILES: O=C1CC(Cc2ccccc2)O1] |
| 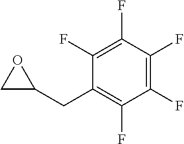 [SMILES: Fc1c(F)c(F)c(CC2CO2)c(F)c1F] | 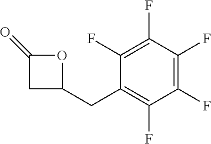 [SMILES: O=C1CC(Cc2c(F)c(F)c(F)c(F)c2F)O1] |
| 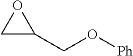 [SMILES: c1ccc(OCC2CO2)cc1] | 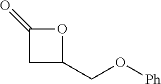 [SMILES: O=C1CC(COc2ccccc2)O1] |
| 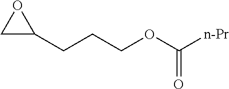 | 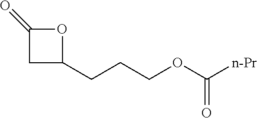 |
| 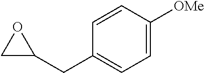 [SMILES: COc1ccc(CC2CO2)cc1] | 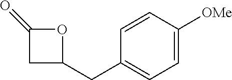 [SMILES: COc1ccc(CC2CC(=O)O2)cc1] |
| 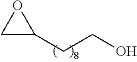 | 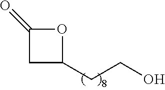 |
| 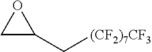 | 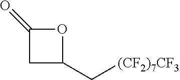 |
| 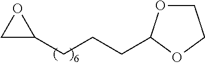 | 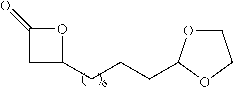 |
| 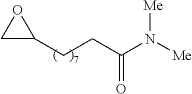 | 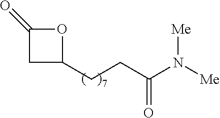 |
| 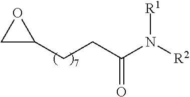 | 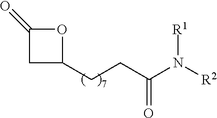 |
| 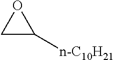 | 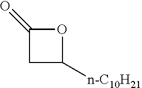 |
| 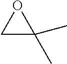 | 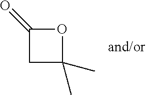 |
| | 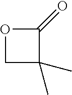 |
| 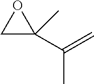 | 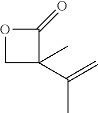 |
| 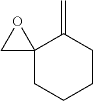 | 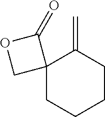 |
| 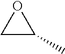 | 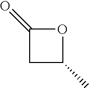 |
| 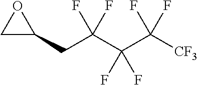 [SMILES: FC(F)(F)C(F)(F)C(F)(F)C(F)(F)C[C@H]1CO1] | 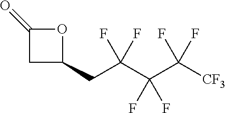 [SMILES: O=C1C[C@H](CC(F)(F)C(F)(F)C(F)(F)C(F)(F)F)O1] |
| 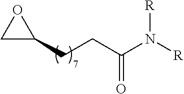 | 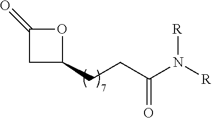 |
| 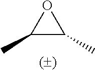 | 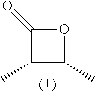 |
| 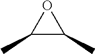 | 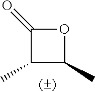 |
| 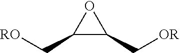 | 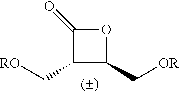 |
| 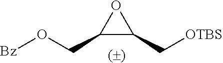 | 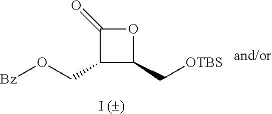 |
| | 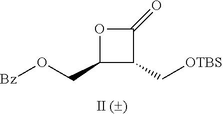 |
| 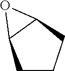 | 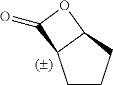 |
| 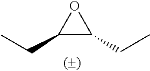 | 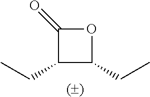 |
| 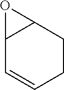 | 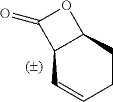 |
| 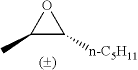 | 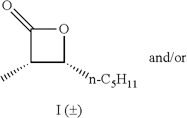 |
| | 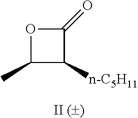 |
| 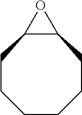 | 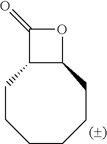 [SMILES: O=C1O[C@H]2CCCCCC[C@H]12] |
| 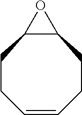 | 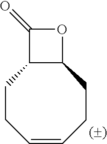 [SMILES: O=C1O[C@H]2CC/C=C\CC[C@H]12] |
| 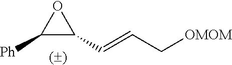 | 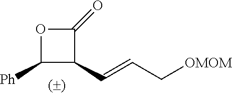 |
| 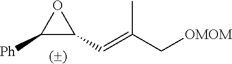 | 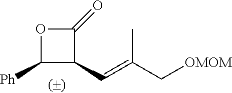 |
| 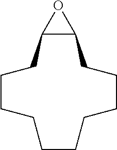 [SMILES: C1CCCCC[C@H]2O[C@H]2CCCC1] | 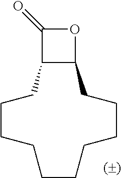 [SMILES: O=C1O[C@H]2CCCCCCCCCC[C@H]12] |
| 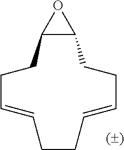 [SMILES: C1=C/CC[C@H]2O[C@@H]2CC/C=C/CC/1] | 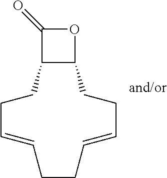 [SMILES: O=C1O[C@@H]2CC/C=C/CC/C=C/CC[C@H]12] |
| | 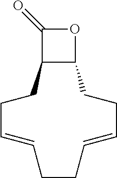 [SMILES: O=C1O[C@@H]2CC/C=C/CC/C=C/CC[C@@H]12] |
| 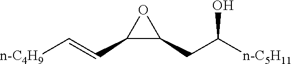 | 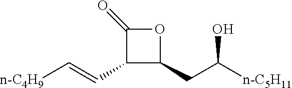 |
| 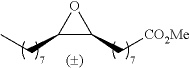 | 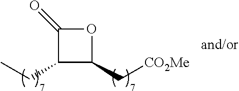 |
| | 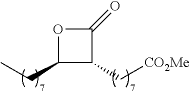 |
| 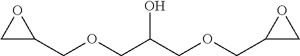 [SMILES: OC(COCC1CO1)COCC1CO1] | 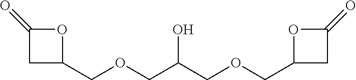 [SMILES: O=C1CC(COCC(O)COCC2CC(=O)O2)O1] |
| 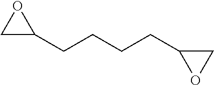 | 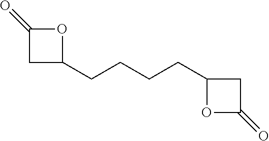 [SMILES: O=C1CC(CCCCC2CC(=O)O2)O1] |
| 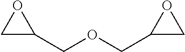 | 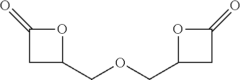 [SMILES: O=C1CC(COCC2CC(=O)O2)O1] |

In preferred embodiments of the present invention, the multifunctional catalysts may include one or more portions having polymerization functionality for producing a polymer. The term “polymer”, as used herein, refers to a molecule of high relative molecular mass, the structure of which comprises the multiple repetitions of units derived, actually or conceptually, from molecules of low relative molecular mass. In certain preferred embodiments, a polymer is comprised of only one monomer species (e.g., an epoxide). In certain other preferred embodiments, a polymer is a copolymer, terpolymer, heteropolymer, block copolymer, or tapered heteropolymer of one or more epoxides.

In preferred embodiments, the processes of the present invention comprise the steps: introducing an epoxide reagent and a carbon monoxide reagent to a reaction vessel; contacting the epoxide reagent and a carbon monoxide reagent with a first portion of a multifunctional catalyst having carbonylation functionality to produce a beta-lactone intermediate; contacting the beta-lactone intermediate with a second portion of the multifunctional catalyst having ring-opening polymerization functionality to produce a polylactone product; and removing the polylactone product from the reaction vessel.

In preferred embodiments, the processes of the present invention provide for ring-opening polymerization. The multifunctional catalyst may have a first portion with a carbonylation functionality and a second portion having ring-opening polymerization functionality. The second portion having ring-opening polymerization functionality may comprise a carboxylate salt of an organic cation, metal-containing complexes, protonated amine, quaternary ammonium salt, guanidinium group, and/or optionally substituted nitrogen-containing heterocycle to name a few. The epoxide reagent and carbon monoxide reagent may be introduced to a reaction vessel and react with the first portion having a carbonylation functionality to produce a beta-lactone intermediate. The beta-lactone may react with the second portion having ring-opening polymerization functionality to produce a polylactone intermediate and/or polylactone product wherein the polylactone intermediate may react with a third portion of the multifunctional catalyst and the polylactone product may be removed from the reaction vessel.

The portions of the multifunctional catalyst suitable for the ring-opening polymerization may include one or more moieties from the following: Journal of the American Chemical Society (2002), 124(51), 15239-15248 Macromolecules, vol. 24, No. 20, pp. 5732-5733, Journal of Polymer Science, Part A-1, vol. 9, No. 10, pp. 2775-2787; Inoue, S., Y. Tomoi, T. Tsuruta & J. Furukawa; Macromolecules, vol. 26, No. 20, pp. 5533-5534; Macromolecules, vol. 23, No. 13, pp. 3206-3212; Polymer Preprints (1999), 40(1), 508-509; Macromolecules, vol. 21, No. 9, pp. 2657-2668; and Journal of Organometallic Chemistry, vol. 341, No. 1-3, pp. 83-9; and in U.S. Pat. Nos. 3,678,069, 3,169,945, 6,133,402; 5,648,452; 6,316,590; 6,538,101; and 6,608,170. The entirety of each of which is hereby incorporated herein by reference. In certain preferred embodiments, a moiety of the portions of the multifunctional catalyst suitable for the ring-opening polymerization may be boned or otherwise tethered to a heterogenous support comprising insoluble siliceous material or an insoluble carbon matrix.

In certain embodiments, polymerization of a beta-lactone intermediate is performed in the presence of polymerization initiator to produce a polylactone product. In some embodiments, the polymerization initiator is an ionic initiator. In variations of this aspect, the ionic initiator has the general formula of M″X where M″ is cationic and X is anionic. M″ is selected from the group consisting of Li+, Na+, K+, Mg2+, Ca2+, and A13+. In some embodiments, M″ is Na+. In some embodiments, M″ is an organic cation. In some embodiments, the organic cation is selected from the group consisting of quaternary ammonium, imidazolium, and bis(triphenylphosphine)iminium. In some embodiments, the quaternary ammonium cation is tetraalkyl ammonium. In some embodiments, M″ is a metal-containing complex and X is a nucleophilic anion. Suitable nucleophilic anions include, but not limited to, compounds comprising at least one carboxylate group, at least one alkoxide group, at least one phenoxide group, and combination thereof. In some embodiments, the nucleophilic anion is selected from the group consisting of halides, hydroxide, alkoxide, carboxylate, and combination thereof. In some embodiments, the ionic initiator is sodium acrylate. In some embodiments, the ionic initiator is tetrabutylammonium acrylate. In certain preferred embodiments, M″ is bonded or otherwise tethered to a heterogenous support comprising insoluble siliceous material or an insoluble carbon matrix.

In certain preferred embodiments, the processes of the present invention provide for the carbonylation of an epoxide reagent with a carbon monoxide reagent to produce a beta-lactone intermediate and for the subsequent co-polymerization of the beta-lactone intermediate with another reagent. In certain preferred embodiments, the processes include a multifunctional catalyst with a first portion having carbonylation functionality and at least a second portion with co-polymerization functionality. In certain embodiments, the beta-lactone intermediate may be co-polymerized with monomers or another polymer having hydroxyl functional groups such as simple alcohols, diols, triols, polyols, and sugar alcohols.

In certain preferred embodiments, the beta-lactone intermediate may be co-polymerized with a cyclic anhydride such as a succinic anhydride intermediate. The co-polymerization of a beta-lactone intermediate and a cyclic anhydride may be performed in the presence of polymerization initiator. In certain preferred embodiments, the processes of the present invention provide for co-polymerization. The multifunctional catalyst may have a first portion with a carbonylation functionality and one or more other portions with co-polymerization functionality. The one or more other portions may comprise condensation polymerization catalysts, oxide catalysts, precious metal catalysts, and/or combinations thereof. In preferred embodiments providing for co-polymerization, one or more reagents may be introduced to the reaction vessel for reaction with a beta-lactone intermediate. For example, a cyclic anhydride reagent may be introduced to the reaction vessel for co-polymerization with a beta-lactone intermediate.

In certain preferred embodiments, an epoxide reagent and carbon monoxide reagent may contact and react with a first portion of a multifunctional catalyst having a carbonylation functionality to produce a beta-lactone intermediate; the beta-lactone intermediate may partially react with the first portion of a multifunctional catalyst having a carbonylation functionality to produce a succinic anhydride intermediate; and the beta-lactone intermediate and succinic anhydride intermediate may contact a second portion of the multifunctional catalyst having co-polymerization functionality to produce a co-polymer product. In certain embodiments, the amount of succinic anhydride intermediate formed may be controlled by introducing a stochiometric excess of the carbon monoxide reagent. In certain embodiments, co-polymerization may be controlled by the introduction of a polymerization initiator, the physical location of the portion having co-polymerization functionality, and/or the stochiometric ratio of the beta-lactone intermediate to the cyclic anhydride.

In preferred embodiments, the processes of the present invention provide for carbonylation to produce a beta-lactone intermediate and for subsequent modification of the beta-lactone intermediate. The processes may include a multifunctional catalyst comprising at least one metal complex having at least one ligand with a cationic moiety of at least two, at least one anionic metal carbonyl compound, and at least one anionic nucleophile. The multifunctional catalyst having at least one metal complex may catalyze carbonylation of an epoxide in the presence of carbon monoxide to form a beta-lactone intermediate and subsequently catalyze modification the beta-lactone intermediate with an alcohol reagent and/or acid reagent. In certain embodiments, the multifunctional catalyst may catalyze carbonylation of an epoxide in the presence of carbon monoxide to form a beta-lactone intermediate and catalyze the subsequent polymerization of the beta-lactones to provide polylactone oligomers. In some embodiments, the multifunctional catalyst may catalyze subsequent copolymerization of the beta-lactones and/or polylactone oligomers with polyols.

In certain preferred embodiments, the processes of the present invention may provide for modification of a beta-lactone with an ammonia reagent to provide an acrylonitrile product. In certain embodiments, a multifunctional catalyst has a first portion with carbonylation functionality and a second portion comprising a dehydration agent chosen from the following: a phosphorous pentoxide, an organophosphorous compound, a carbodiimide compound, a triazine compound, an organosilicon compound, a transition metal complex, an aluminum complex, or a mixture thereof.

In certain preferred embodiments, the portions of the multifunctional catalyst suitable for modification of a beta-lactone intermediate may include metal, metal oxides, metal nitrides wherein the metal may be chosen from the the group including Cr, Al, V, Mn, Fe, Mo, Sn, Bi, As, Sb, Si, Ce, Zn, Zr, and U. In certain preferred embodiments, the portion is supported such as on silica. Certain examples of a portion of the multifunctional catalyst with modification functionality useful for modification of a beta-lactone intermediate include Cr2O3/Al2O3, KNaMoP/Al2O3, NaMo/Al2O3, AsFeO, SbSnO, FeBiPO, BiMoO, MoO3, MoO3/SiO2, and/or NaMo/Al2O3. In certain embodiments, a portion of the heterogenous catalyst with modification functionality may include a phosphorous pentoxide, an organophosphorous compound, a carbodiimide compound, a triazine compound, an organosilicon compound, a transition metal complex, an aluminum complex, or a mixture thereof.

In certain preferred embodiments, the multifunctional catalysts of the present invention may be formed in one reaction vessel. In some embodiments, the catalysts of the present invention may be formed from catalyst reagents dissolved in solution such as a THF solvent or hexane solvent. In some embodiments, the catalysts of the present invention may be formed by pyrolyzing catalyst reagents in a reaction vessel. In some embodiments, the formation of the catalysts and/or catalyst reagents may include the chemical or electrochemical reduction of metal halides. In some embodiments, metal, alloy, metal alkyls, metal hydrides, complex metal hydrides, metal anhydrides, or mixtures may be reducing agents. In some embodiments, hydrogen or carbon monoxide may be used as a reducing agent. In some embodiments, a reducing agent may contain a sulfur atom.

In preferred embodiments, the multifunctional catalysts of the present invention may include at least one metal complex with the formula [(L^{c})ₐM_{b}]^{Z+}. The L^{c} is the ligand that includes at least two cationic moieties. If two or more L^{c} are present, then each may be the same or different. The M is a metal atom. If two or more M are present, then each may be the same or different. The a is an integer from 1 to 4 inclusive; the b is an integer from 1 to 2 inclusive; and the z is an integer greater than 2 that represents the cationic charge on the metal complex. The metal atom may be chosen from titanium, chromium, manganese, iron, ruthenium, cobalt, rhodium, samarium, rhenium, iridium, zirconium, nickel, palladium, copper, zinc, magnesium, aluminum, gallium, tin, indium, molybdenum, and tungsten.

In preferred embodiments, the multifunctional catalyst may comprise a metal complex having at least one tetradentate ligand such as porphyrin. In certain preferred embodiments, the tetradentate ligand may be a salen ligand. In certain embodiments, the salen ligand may include salicylaldehyde and ethylenediamine molecular parts. In certain embodiments, the metal complex includes a cobalt atom. In certain embodiments, the metal complex may be reacted with divinylbenzene to form an insoluble resin which may function as a heterogenous catalyst.

In certain preferred embodiments, the multifunctional catalyst comprises at least two metal complexes wherein each metal complex includes a metal atom chosen from the group: titanium, chromium, manganese, iron, ruthenium, cobalt, rhodium, samarium, rhenium, iridium, zirconium, nickel, palladium, copper, zinc, magnesium, aluminum, gallium, tin, indium, molybdenum, and tungsten. In certain preferred embodiments, the multifunctional catalyst comprising at least two metal complexes may further comprise a first metal complex having a different concentration than a second metal complex based on the total weight of the multifunctional catalyst. In some embodiments, the second metal complex may be present in an amount from 0.1 to 99 wt. %, e.g., from 0.1 to 95 wt. %, or from 0.1 to 90 wt. %. In some embodiments, the second metal complex may be present in the multifunctional catalyst in an amount from 0.1 to 90 wt. %, e.g. from 0.1 to 80 wt. %, or from 0.1 to75 wt. %. In some embodiments, the second metal complex may be present in an amount from 0.1 to 75 wt. %, e.g., from 0.1 to 70 wt. %, or from 0.1 to 50 wt. %. In some embodiments, the second metal complex may be present in an amount from 0.1 to 50 wt. %, e.g., from 0.1 to 25 wt. %, or from 0.1 to 10 wt. %. In some embodiments, the second metal complex may be present in an amount from 0.1 to 10 wt. %, e.g., from 0.1 to 5 wt. % or from 0.1 to 1 wt. %.

In certain preferred embodiments of the present invention, the multifunctional catalyst includes at least two metal complexes and one or more bridging ligands. The at least two metal complexes further comprises a first metal complex and a second metal complex. A portion of the one or more bridging ligands may be covalently or coordinately bonded to the first metal complex and the second metal complex for tethering the first metal complex to the second metal complex. Bridging ligands may comprise C1-20 aliphatic groups and/or C1-20 heteroaliphatic having 1-10 heteroatoms independently selected from the group consisting of nitrogen, boron, oxygen, and sulfur. In some embodiments, the bridging ligands comprise 6- to 10-membered aryl group and/or 5- to 10-membered heteroaryl having 1-4 heteroatoms independently selected from nitrogen, oxygen, and sulfur. In some embodiment, the bridging ligands comprise 4- to 7-membered heterocyclic having 1-2 heteroatoms independently selected from the group consisting of nitrogen, boron, oxygen, and sulfur. In certain embodiments, bridging ligands provide for close proximity between two portions of the multifunctional catalysts having different functionalities.

Embodiments of the present invention may include one or more portions of the multifunctional catalyst that are bonded or otherwise tethered to a heterogenous support. In certain embodiments, the heterogenous support comprises a siliceous based material, e.g., silica, and/or a carbon based material, e.g., carbon black or activated carbon, although any of a variety of other suitable supporting materials may be used. The heterogenous support material may be selected from the group comprising silica, magnesia, alumina, titania, silica/alumina, pyrogenic silica, high purity silica, zirconia, zincate, carbon (e.g., carbon black or activated carbon), zeolites and mixtures thereof. In certain embodiments, the heterogenous support comprises a polymer. In certain embodiments, the heterogenous support includes one or more metal complexes. Preferably, the heterogenous support comprises a siliceous material such as silica, pyrogenic silica, or high purity silica. In some embodiments, the heterogenous support siliceous material is substantially free of alkaline earth metals, such as magnesium and calcium. In some embodiments, the heterogenous support is present in an amount from 25 wt. % to 99 wt. %, e.g., from 30 wt. % to 98 wt. % or from 35 wt. % to 95 wt. %, based on the total weight of the multifunctional catalyst.

In preferred embodiments, the heterogenous support comprises a siliceous material, e.g., silica, having a surface area of at least 50 m²/g, e.g., at least 100 m²/g, or at least 150 m²/g. In terms of ranges, the siliceous support material preferably has a surface area from 50 to 800 m²/g, e.g., from 100 to 500 m²/g or from 100 to 300 m²/g. High surface area silica, as used throughout the application, refers to silica having a surface area of at least 250 m²/g. For purposes of the present specification, surface area refers to BET nitrogen surface area, meaning the surface area as determined by ASTM D6556-04, the entirety of which is incorporated herein by reference.

The preferred siliceous material also preferably has an average pore diameter from 5 to 100 nm, e.g., from 5 to 50 nm, from 5 to 25 nm or from 5 to 10 nm, as determined by mercury intrusion porosimetry, and an average pore volume from 0.5 to 3.0 cm³/g, e.g., from 0.7 to 2 cm³/g or from 0.8 to 1.5 cm³/g, as determined by mercury intrusion porosimetry.

In some embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the siliceous support material has a morphology that allows for a packing density from 0.1 to 2.0 g/cm³, e.g., from 0.2 to 1.5 g/cm³ or from 0.3 to 0.5 g/cm³.

In some embodiments, one or more portions of the multifunctional catalyst may be impregnated into the heterogenous support. With impregnation, a moiety of at least one portion and heterogenous support material are mixed together followed by drying and calcination to form the final multifunctional catalyst with heterogenous support. With simultaneous impregnation, it may be desired to employ a dispersion agent, surfactant, or solubilizing agent, e.g., ammonium oxalate or an acid such as acetic or nitric acid, to facilitate the dispersing or solubilizing of the first, second and/or optional third metal complex in the event one or more of the metal complexes are incompatible.

With sequential impregnation, a first portion may be first added to the heterogenous support material followed by drying and calcining, and the resulting material may then be impregnated by subsequent one or more portions followed by an additional drying and calcining to form the final multifunctional catalyst with heterogenous support. In some embodiments, additional portions may be added either with the first and/or second portion or in a separate sequential impregnation, followed by drying and calcination. In some embodiments, combinations of sequential and simultaneous impregnation may be employed if desired.

In preferred embodiments, the multifunctional catalyst may be employed in a fixed bed reactor where the reactants are passed over or through the catalyst. Other reactors, such as fluid or ebullient bed reactors, can be employed. In some instances, the multifunctional catalysts may be used in conjunction with an inert material to regulate the pressure drop of the reactant stream through the catalyst bed and the contact time of the reactant compounds with the multifunctional catalyst.

What is claimed is:

1. A process for producing a product with a multifunctional catalyst comprising:
a. introducing at least an epoxide reagent and a carbon monoxide reagent to a reaction vessel containing a multifunctional catalyst;
b. contacting a first portion of the multifunctional catalyst having carbonylation functionality with at least the epoxide reagent and carbon monoxide reagent to produce one or more intermediates;
c. contacting a second portion of the multifunctional catalyst with the one or more intermediates to produce a product;
d. removing the product from the reaction vessel.

2. The process from claim 1, wherein the at least one epoxide reagent includes an ethylene oxide reagent.

3. The process from claim 1, wherein the one or more intermediates includes a beta-lactone intermediate.

4. The process from claim 1, wherein the one or more intermediates includes a succinic anhydride intermediate.

5. The process from claim 1, wherein the first portion of the multifunctional catalyst comprises a metal carbonyl-Lewis acid.

6. The process from claim 1, wherein the product includes succinic anhydride.

7. The process from claim 1, wherein the second portion of the multifunctional catalyst has a ring-opening polymerization functionality.

8. The process from claim 1, wherein the second portion of the multifunctional catalyst includes a polymerization initiator.

9. The process from claim 1, wherein the second portion of the multifunctional catalyst has modification functionality useful for modification of a beta-lactone intermediate.

10. The process from claim 9, wherein the second portion comprises Cr2O3/Al2O3, KNaMoP/Al2O3, NaMo/Al2O3, AsFeO, SbSnO, FeBiPO, BiMoO, MoO3, MoO3/SiO2, and/or NaMo/Al2O3

11. The process from claim 1, wherein the product is a polylactone homopolymer comprising beta-propiolactone monomers.

12. The process from claim 1, wherein the product is a polylactone heteropolymer comprising a beta-propiolactone monomer and one or more other beta-lactone monomers.

13. The process from claim 1, wherein the product is a co-polymer comprising a beta-propiolactone monomer and a succinic anhydride monomer.

14. The process from claim 1, wherein the multifunctional catalyst comprises a heterogenous support.

15. The process from claim 1, wherein the multifunctional catalyst comprises a metal carbonyl-Lewis acid and a heterogenous support.

16. The process from claim 1, wherein the multifunctional catalyst comprises metal carbonyl-Lewis acid, and ionic initiator, and a heterogenous support.

17. The process form claim 5, wherein the process further includes:
a. introducing an ammonia reagent to the reaction vessel; and
b. contacting the beta-lactone intermediate and the ammonia reagent with the second portion of the multifunctional catalyst to produce an acrylonitrile product, wherein the second portion of the multifunctional catalyst includes a moiety comprising a dehydration agent chosen from the following: a phosphorous pentoxide, an organophosphorous compound, a carbodiimide compound, a triazine compound, an organosilicon compound, a transition metal complex, an aluminum complex, or a mixture thereof.

18. The process form claim 17, wherein the process further comprises a step for contacting the acrylonitrile product with a third portion of the multifunctional catalyst to produce polyacrylonitrile, wherein the third portion comprising a metal complex bonded to a heterogenous support.

19. The process from claim 18, wherein the metal complex comprises a metal chosen from Ti, Cr, Mn, Fe, Ru, Co, Rh, Sm, Re, Ir, Zr, Ni, Pd, Co, Zn, Mg, Al, Ga, Sn, In, Mo, W.
